(19) United States
(12) Patent Application Publication
(10) Pub. No.: US 2022/0185062 A1
(43) Pub. Date: Jun. 16, 2022

(54) Cleaning Vehicle Cabins Using Cabin Pressure And Controlled Airflow

(71) Applicant: Motional AD LLC, Boston, MA (US)

(72) Inventors: Adam-Ridgely Khaw, Roslindale, MA (US); Linh Pham, Cambridge, MA (US); Tristan Dwyer, Stow, MA (US); Christopher Konopka, Peabody, MA (US)

(21) Appl. No.: 17/118,480

(22) Filed: Dec. 10, 2020

(51) Int. Cl. B60H 1/00; B60N 2/00; B60Q 3/68; B60S 1/64; B60H 3/00; A61L 9/014; A61L 9/20

(52) U.S. Cl. CPC B60H 1/00742; B60N 2/002; B60Q 3/68; B60S 1/64; B60H 3/0078; G01J 5/10; B60H 1/00849; B60H 1/00985; A61L 9/014; A61L 9/20; B60H 1/008

(57) ABSTRACT

Among other things, vehicle cabin pressure systems and vehicle cabin pressure techniques are described for a vehicle. The vehicle includes a cabin configured to seat a plurality of passengers; at least one inlet including at least one inlet fan; at least one occupancy sensor; and at least one processor configured to execute computer executable instructions, the execution carrying out operations including: receiving a signal from the at least one occupancy sensor indicating a number of passengers within the cabin; and controlling the at least one inlet to cause air that is or has been filtered to flow into the cabin and increase a pressure in the cabin to a predetermined level above an ambient pressure level outside the vehicle based on the number of passengers in the cabin.

### FIELD OF THE INVENTION

This description relates to the removal of airborne particulates in vehicle cabins.

### BACKGROUND

Passengers within a vehicle cabin are oftentimes exposed to airborne particulates (e.g., dust, pollen, smoke, soot, smog) which include allergens, bacteria, and viruses. This exposure is exacerbated for shared vehicles, such as taxis, buses and the like. Conventional cleaning methods of applying chemical disinfectants to vehicle surfaces can rid surfaces of settled particulates. However, even a brief exposure to an airborne particulate can infect a passenger or impact their comfort.

### BRIEF DESCRIPTION OF THE DRAWINGS

FIG. 1 shows an example of an autonomous vehicle having autonomous capability.

FIG. 2 shows an example architecture for an autonomous vehicle.

FIG. 3 shows a side view of a vehicle with a cabin pressure system.

FIG. 4 shows a top schematic view of the vehicle with the cabin pressure system.

FIG. 5 shows the vehicle in network communication with a mobile device.

FIG. 6 shows the vehicle with at least one audible sensor.

FIG. 7 shows the vehicle with at least one ultraviolet light source.

FIG. 8 shows a schematic view of a vehicle with four pressurized compartments.

FIG. 9 shows a schematic of the components of a cabin pressure system.

FIG. 10 shows a flow chart of a first method of a cabin pressure system.

FIG. 11 shows a flow chart of a second method of a cabin pressure system.

FIG. 12 shows a flow chart of a third method of a cabin pressure system.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

In the drawings, specific arrangements or orderings of schematic elements, such as those representing devices, modules, instruction blocks and data elements, are shown for ease of description. However, it should be understood by those skilled in the art that the specific ordering or arrangement of the schematic elements in the drawings is not meant to imply that a particular order or sequence of processing, or separation of processes, is required. Further, the inclusion of a schematic element in a drawing is not meant to imply that such element is required in all embodiments or that the features represented by such element may not be included in or combined with other elements in some embodiments.

Further, in the drawings, where connecting elements, such as solid or dashed lines or arrows, are used to illustrate a connection, relationship, or association between or among two or more other schematic elements, the absence of any such connecting elements is not meant to imply that no connection, relationship, or association can exist. In other words, some connections, relationships, or associations between elements are not shown in the drawings so as not to obscure the disclosure. In addition, for ease of illustration, a single connecting element is used to represent multiple connections, relationships or associations between elements. For example, where a connecting element represents a communication of signals, data, or instructions, it should be understood by those skilled in the art that such element represents one or multiple signal paths (e.g., a bus), as may be needed, to affect the communication.

Reference will now be made in detail to embodiments, examples of which are illustrated in the accompanying drawings. In the following detailed description, numerous specific details are set forth in order to provide a thorough understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

Several features are described hereafter that can each be used independently of one another or with any combination of other features. However, any individual feature may not address any of the problems discussed above or might only address one of the problems discussed above. Some of the problems discussed above might not be fully addressed by any of the features described herein. Although headings are provided, information related to a particular heading, but not found in the section having that heading, may also be found elsewhere in this description. Embodiments are described herein according to the following outline:

1. General Overview

2. System Overview

3. Autonomous Vehicle Architecture

4. Autonomous Vehicle Inputs

5. Vehicle Cabin Pressure Systems

### General Overview

Passengers within a vehicle are oftentimes sensitive to airborne particulates (e.g., dust, pollen, smoke, soot, smog) which include allergens, bacteria, and viruses. Minimizing the levels of particulates within a vehicle cabin helps reduce the spread of bacteria and viruses and also helps increase the comfort level of the passengers (e.g., reduced allergens). Reducing the level of airborne particulates also increases the perceived cleanliness of the air (e.g., passengers notice when smoke is present). When the vehicle cabin is positively pressurized slightly above the ambient pressure outside the vehicle, the air inside the cabin, as well as the airborne particulates, flow out of the cabin due to the high/low pressure gradient.

In an embodiment, the positive pressurization of the vehicle cabin depends on conditions within the vehicle. In some examples, the pressurization of the vehicle cabin is based on whether zero passengers are present within the vehicle (e.g., to perform a clean before the passengers enter and/or after the passengers exit). In some examples, the pressurization of the vehicle cabin is based on whether one of the passengers sneezes or coughs (e.g., to trigger the pressurization process immediately). In some examples, the pressurization of the vehicle cabin is based on the current vehicle speed (e.g., to determine if it makes sense to open the vents to allow roadway air into the cabin). In some examples, the pressurization is based on passenger comfort preferences (e.g., are they particularly sensitive to allergens, of high risk for illness, and/or are they sensitive to pressurized environments). In an embodiment, the system controls the pressurization system to run in reverse to “vacuum” the air within the cabin out of the vehicle. In an embodiment, the vehicle cabin is compartmentalized so each passenger has their own airflow and associated cleanliness/comfort settings. In an embodiment, a UV light (and in particular a far-UVC light) is used to reduce a bacteria level of the cabin air.

In some examples, a pressurized cabin that is controlled by at least one processor of the vehicle that receives information on the passengers and passenger preferences gives a more comfortable and cleaner passenger environment compared to a traditional vehicle. For example, a system that incorporates user preferences enables a pressurized system to be tailored to the passengers of the vehicle.

In some examples, a system that includes a sensor to detect when a passenger coughs or sneezes enables the system to clean on demand and clean airborne particulates before the airborne particulates stick to surfaces. Having a system configured to run in reverse to vacuum out the vehicle allows the system to clean even non-airborne particles.

### System Overview

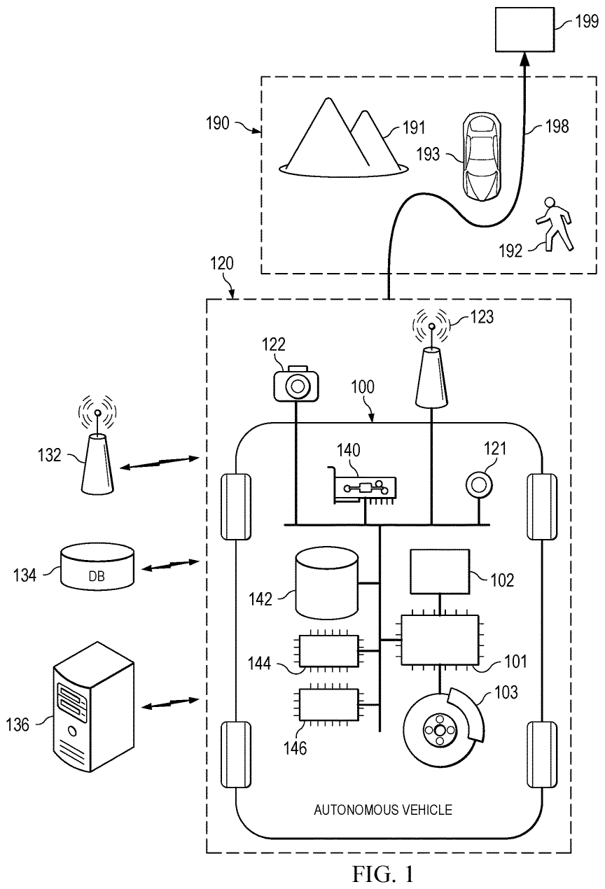

FIG. 1 shows an example of an autonomous vehicle 100 having autonomous capability.

As used herein, the term “autonomous capability” refers to a function, feature, or facility that enables a vehicle to be partially or fully operated without real-time human intervention, including without limitation fully autonomous vehicles, highly autonomous vehicles, and conditionally autonomous vehicles.

As used herein, an autonomous vehicle (AV) is a vehicle that possesses autonomous capability.

As used herein, “vehicle” includes means of transportation of goods or people. For example, cars, buses, trains, airplanes, drones, trucks, boats, ships, submersibles, dirigibles, etc. A driverless car is an example of a vehicle.

As used herein, “trajectory” refers to a path or route to navigate an AV from a first spatiotemporal location to second spatiotemporal location. In an embodiment, the first spatiotemporal location is referred to as the initial or starting location and the second spatiotemporal location is referred to as the destination, final location, goal, goal position, or goal location. In some examples, a trajectory is made up of one or more segments (e.g., sections of road) and each segment is made up of one or more blocks (e.g., portions of a lane or intersection). In an embodiment, the spatiotemporal locations correspond to real world locations. For example, the spatiotemporal locations are pick up or drop-off locations to pick up or drop-off persons or goods.

As used herein, “sensor(s)” includes one or more hardware components that detect information about the environment surrounding the sensor. Some of the hardware components can include sensing components (e.g., image sensors, biometric sensors), transmitting and/or receiving components (e.g., laser or radio frequency wave transmitters and receivers), electronic components such as analog-to-digital converters, a data storage device (such as a RAM and/or a nonvolatile storage), software or firmware components and data processing components such as an ASIC (application-specific integrated circuit), a microprocessor and/or a microcontroller.

As used herein, a “scene description” is a data structure (e.g., list) or data stream that includes one or more classified or labeled objects detected by one or more sensors on the AV vehicle or provided by a source external to the AV.

As used herein, a “road” is a physical area that can be traversed by a vehicle, and may correspond to a named thoroughfare (e.g., city street, interstate freeway, etc.) or may correspond to an unnamed thoroughfare (e.g., a driveway in a house or office building, a section of a parking lot, a section of a vacant lot, a dirt path in a rural area, etc.). Because some vehicles (e.g., 4-wheel-drive pickup trucks, sport utility vehicles, etc.) are capable of traversing a variety of physical areas not specifically adapted for vehicle travel, a “road” may be a physical area not formally defined as a thoroughfare by any municipality or other governmental or administrative body.

As used herein, a “lane” is a portion of a road that can be traversed by a vehicle. A lane is sometimes identified based on lane markings. For example, a lane may correspond to most or all of the space between lane markings, or may correspond to only some (e.g., less than 50%) of the space between lane markings. For example, a road having lane markings spaced far apart might accommodate two or more vehicles between the markings, such that one vehicle can pass the other without traversing the lane markings, and thus could be interpreted as having a lane narrower than the space between the lane markings, or having two lanes between the lane markings. A lane could also be interpreted in the absence of lane markings. For example, a lane may be defined based on physical features of an environment, e.g., rocks and trees along a thoroughfare in a rural area or, e.g., natural obstructions to be avoided in an undeveloped area. A lane could also be interpreted independent of lane markings or physical features. For example, a lane could be interpreted based on an arbitrary path free of obstructions in an area that otherwise lacks features that would be interpreted as lane boundaries. In an example scenario, an AV could interpret a lane through an obstruction-free portion of a field or empty lot. In another example scenario, an AV could interpret a lane through a wide (e.g., wide enough for two or more lanes) road that does not have lane markings. In this scenario, the AV could communicate information about the lane to other AVs so that the other AVs can use the same lane information to coordinate path planning among themselves.

“One or more” includes a function being performed by one element, a function being performed by more than one element, e.g., in a distributed fashion, several functions being performed by one element, several functions being performed by several elements, or any combination of the above.

It will also be understood that, although the terms first, second, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

The terminology used in the description of the various described embodiments herein is for the purpose of describing particular embodiments only and is not intended to be limiting. As used in the description of the various described embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this description, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, an AV system refers to the AV along with the array of hardware, software, stored data, and data generated in real-time that supports the operation of the AV. In an embodiment, the AV system is incorporated within the AV. In an embodiment, the AV system is spread across several locations. For example, some of the software of the AV system is implemented on a cloud computing environment similar to a cloud computing environment.

In general, this document describes technologies applicable to any vehicles that have one or more autonomous capabilities including fully autonomous vehicles, highly autonomous vehicles, and conditionally autonomous vehicles, such as so-called Level 5, Level 4 and Level 3 vehicles, respectively (see SAE International's standard J3016: Taxonomy and Definitions for Terms Related to On-Road Motor Vehicle Automated Driving Systems, which is incorporated by reference in its entirety, for more details on the classification of levels of autonomy in vehicles). The technologies described in this document are also applicable to partially autonomous vehicles and driver assisted vehicles, such as so-called Level 2 and Level 1 vehicles (see SAE International's standard J3016: Taxonomy and Definitions for Terms Related to On-Road Motor Vehicle Automated Driving Systems). In an embodiment, one or more of the Level 1, 2, 3, 4 and 5 vehicle systems may automate certain vehicle operations (e.g., steering, braking, and using maps) under certain operating conditions based on processing of sensor inputs. The technologies described in this document can benefit vehicles in any levels, ranging from fully autonomous vehicles to human-operated vehicles.

Autonomous vehicles have advantages over vehicles that require a human driver. One advantage is safety. For example, in 2016, the United States experienced 6 million automobile accidents, 2.4 million injuries, 40,000 fatalities, and 13 million vehicles in crashes, estimated at a societal cost of $910+ billion. U.S. traffic fatalities per 100 million miles traveled have been reduced from about six to about one from 1965 to 2015, in part due to additional safety measures deployed in vehicles. For example, an additional half second of warning that a crash is about to occur is believed to mitigate 60% of front-to-rear crashes. However, passive safety features (e.g., seat belts, airbags) have likely reached their limit in improving this number. Thus, active safety measures, such as automated control of a vehicle, are the likely next step in improving these statistics. Because human drivers are believed to be responsible for a critical pre-crash event in 95% of crashes, automated driving systems are likely to achieve better safety outcomes, e.g., by reliably recognizing and avoiding critical situations better than humans; making better decisions, obeying traffic laws, and predicting future events better than humans; and reliably controlling a vehicle better than a human.

Referring to FIG. 1, an AV system 120 operates the AV 100 along a trajectory 198 through an environment 190 to a destination 199 (sometimes referred to as a final location) while avoiding objects (e.g., natural obstructions 191, vehicles 193, pedestrians 192, cyclists, and other obstacles) and obeying rules of the road (e.g., rules of operation or driving preferences).

In an embodiment, the AV system 120 includes devices 101 that are instrumented to receive and act on operational commands from the computer processors 146. We use the term “operational command” to mean an executable instruction (or set of instructions) that causes a vehicle to perform an action (e.g., a driving maneuver or movement). Operational commands can, without limitation, including instructions for a vehicle to start moving forward, stop moving forward, start moving backward, stop moving backward, accelerate, decelerate, perform a left turn, and perform a right turn. Examples of devices 101 include a steering control 102, brakes 103, gears, accelerator pedal or other acceleration control mechanisms, windshield wipers, side-door locks, window controls, and turn-indicators.

In an embodiment, the AV system 120 includes sensors 121 for measuring or inferring properties of state or condition of the AV 100, such as the AV's position, linear and angular velocity and acceleration, and heading (e.g., an orientation of the leading end of AV 100). Example of sensors 121 are GPS, inertial measurement units (IMU) that measure both vehicle linear accelerations and angular rates, wheel speed sensors for measuring or estimating wheel slip ratios, wheel brake pressure or braking torque sensors, engine torque or wheel torque sensors, and steering angle and angular rate sensors.

In an embodiment, the sensors 121 also include sensors for sensing or measuring properties of the AV's environment. For example, monocular or stereo video cameras 122 in the visible light, infrared or thermal (or both) spectra, LiDAR 123, RADAR, ultrasonic sensors, time-of-flight (TOF) depth sensors, speed sensors, temperature sensors, humidity sensors, and precipitation sensors.

In an embodiment, the AV system 120 includes a data storage unit 142 and memory 144 for storing machine instructions associated with computer processors 146 or data collected by sensors 121. In an embodiment, the data storage unit 142 and memory 144 store historical, real-time, and/or predictive information about the environment 190. In an embodiment, the stored information includes maps, driving performance, traffic congestion updates or weather conditions. In an embodiment, data relating to the environment 190 is transmitted to the AV 100 via a communications channel from a remotely located database 134.

In an embodiment, the AV system 120 includes communications devices 140 for communicating measured or inferred properties of other vehicles' states and conditions, such as positions, linear and angular velocities, linear and angular accelerations, and linear and angular headings to the AV 100. These devices include Vehicle-to-Vehicle (V2V) and Vehicle-to-Infrastructure (V2I) communication devices and devices for wireless communications over point-to-point or ad hoc networks or both. In an embodiment, the communications devices 140 communicate across the electromagnetic spectrum (including radio and optical communications) or other media (e.g., air and acoustic media). A combination of Vehicle-to-Vehicle (V2V) Vehicle-to-Infrastructure (V2I) communication (and, in some embodiments, one or more other types of communication) is sometimes referred to as Vehicle-to-Everything (V2X) communication. V2X communication typically conforms to one or more communications standards for communication with, between, and among autonomous vehicles.

In an embodiment, the communication devices 140 include communication interfaces. For example, wired, wireless, WiMAX, Wi-Fi, Bluetooth, satellite, cellular, optical, near field, infrared, or radio interfaces. The communication interfaces transmit data from a remotely located database 134 to AV system 120. In an embodiment, the remotely located database 134 is embedded in a cloud computing environment. The communication interfaces 140 transmit data collected from sensors 121 or other data related to the operation of AV 100 to the remotely located database 134. In an embodiment, communication interfaces 140 transmit information that relates to teleoperations to the AV 100. In some embodiments, the AV 100 communicates with other remote (e.g., “cloud”) servers 136.

In an embodiment, the remotely located database 134 also stores and transmits digital data (e.g., storing data such as road and street locations). Such data is stored on the memory 144 on the AV 100, or transmitted to the AV 100 via a communications channel from the remotely located database 134.

In an embodiment, the remotely located database 134 stores and transmits historical information about driving properties (e.g., speed and acceleration profiles) of vehicles that have previously traveled along trajectory 198 at similar times of day. In one implementation, such data may be stored on the memory 144 on the AV 100, or transmitted to the AV 100 via a communications channel from the remotely located database 134.

Computing devices 146 located on the AV 100 algorithmically generate control actions based on both real-time sensor data and prior information, allowing the AV system 120 to execute its autonomous driving capabilities.

In an embodiment, the AV system 120 includes computer peripherals 132 coupled to computing devices 146 for providing information and alerts to, and receiving input from, a user (e.g., an occupant or a remote user) of the AV 100. The coupling is wireless or wired. Any two or more of the interface devices may be integrated into a single device.

In an embodiment, the AV system 120 receives and enforces a privacy level of a passenger, e.g., specified by the passenger or stored in a profile associated with the passenger. The privacy level of the passenger determines how particular information associated with the passenger (e.g., passenger comfort data, biometric data, etc.) is permitted to be used, stored in the passenger profile, and/or stored on the cloud server 136 and associated with the passenger profile. In an embodiment, the privacy level specifies particular information associated with a passenger that is deleted once the ride is completed. In an embodiment, the privacy level specifies particular information associated with a passenger and identifies one or more entities that are authorized to access the information. Examples of specified entities that are authorized to access information can include other AVs, third party AV systems, or any entity that could potentially access the information.

A privacy level of a passenger can be specified at one or more levels of granularity. In an embodiment, a privacy level identifies specific information to be stored or shared. In an embodiment, the privacy level applies to all the information associated with the passenger such that the passenger can specify that none of her personal information is stored or shared. Specification of the entities that are permitted to access particular information can also be specified at various levels of granularity. Various sets of entities that are permitted to access particular information can include, for example, other AVs, cloud servers 136, specific third party AV systems, etc.

In an embodiment, the AV system 120 or the cloud server 136 determines if certain information associated with a passenger can be accessed by the AV 100 or another entity. For example, a third-party AV system that attempts to access passenger input related to a particular spatiotemporal location must obtain authorization, e.g., from the AV system 120 or the cloud server 136, to access the information associated with the passenger. For example, the AV system 120 uses the passenger's specified privacy level to determine whether the passenger input related to the spatiotemporal location can be presented to the third-party AV system, the AV 100, or to another AV. This enables the passenger's privacy level to specify which other entities are allowed to receive data about the passenger's actions or other data associated with the passenger.

### Autonomous Vehicle Architecture

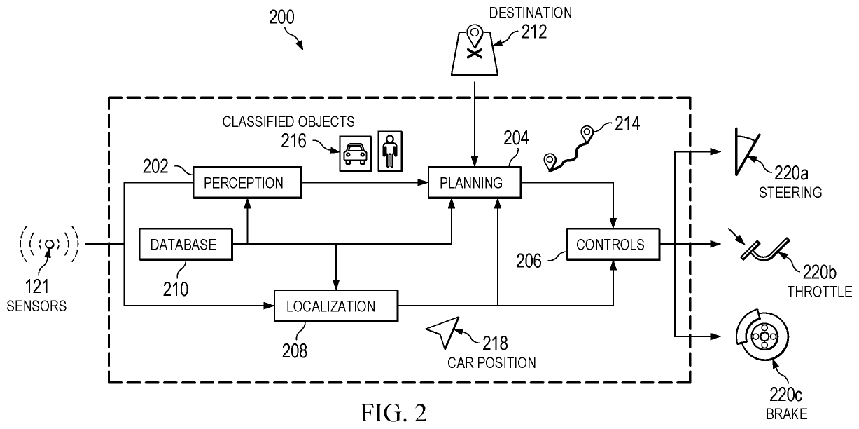

FIG. 2 shows an example architecture 200 for an autonomous vehicle (e.g., the AV 100 shown in FIG. 1). The architecture 200 includes a perception module 202 (sometimes referred to as a perception circuit), a planning module 204 (sometimes referred to as a planning circuit), a control module 206 (sometimes referred to as a control circuit), a localization module 208 (sometimes referred to as a localization circuit), and a database module 210 (sometimes referred to as a database circuit). Each module plays a role in the operation of the AV 100. Together, the modules 202, 204, 206, 208, and 210 may be part of the AV system 120 shown in FIG. 1. In some embodiments, any of the modules 202, 204, 206, 208, and 210 is a combination of computer software (e.g., executable code stored on a computer-readable medium) and computer hardware (e.g., one or more microprocessors, microcontrollers, application-specific integrated circuits [ASICs]), hardware memory devices, other types of integrated circuits, other types of computer hardware, or a combination of any or all of these things). Each of the modules 202, 204, 206, 208, and 210 is sometimes referred to as a processing circuit (e.g., computer hardware, computer software, or a combination of the two). A combination of any or all of the modules 202, 204, 206, 208, and 210 is also an example of a processing circuit.

In use, the planning module 204 receives data representing a destination 212 and determines data representing a trajectory 214 (sometimes referred to as a route) that can be traveled by the AV 100 to reach (e.g., arrive at) the destination 212. In order for the planning module 204 to determine the data representing the trajectory 214, the planning module 204 receives data from the perception module 202, the localization module 208, and the database module 210.

The perception module 202 identifies nearby physical objects using one or more sensors 121, e.g., as also shown in FIG. 1. The objects are classified (e.g., grouped into types such as pedestrian, bicycle, automobile, traffic sign, etc.) and a scene description including the classified objects 216 is provided to the planning module 204.

The planning module 204 also receives data representing the AV position 218 from the localization module 208. The localization module 208 determines the AV position by using data from the sensors 121 and data from the database module 210 (e.g., a geographic data) to calculate a position. For example, the localization module 208 uses data from a GNSS (Global Navigation Satellite System) sensor and geographic data to calculate a longitude and latitude of the AV. In an embodiment, data used by the localization module 208 includes high-precision maps of the roadway geometric properties, maps describing road network connectivity properties, maps describing roadway physical properties (such as traffic speed, traffic volume, the number of vehicular and cyclist traffic lanes, lane width, lane traffic directions, or lane marker types and locations, or combinations of them), and maps describing the spatial locations of road features such as crosswalks, traffic signs or other travel signals of various types. In an embodiment, the high-precision maps are constructed by adding data through automatic or manual annotation to low-precision maps.

The control module 206 receives the data representing the trajectory 214 and the data representing the AV position 218 and operates the control functions 220a-c (e.g., steering, throttling, braking, ignition) of the AV in a manner that will cause the AV 100 to travel the trajectory 214 to the destination 212. For example, if the trajectory 214 includes a left turn, the control module 206 will operate the control functions 220a-c in a manner such that the steering angle of the steering function will cause the AV 100 to turn left and the throttling and braking will cause the AV 100 to pause and wait for passing pedestrians or vehicles before the turn is made.

### Vehicle Cabin Pressure Systems

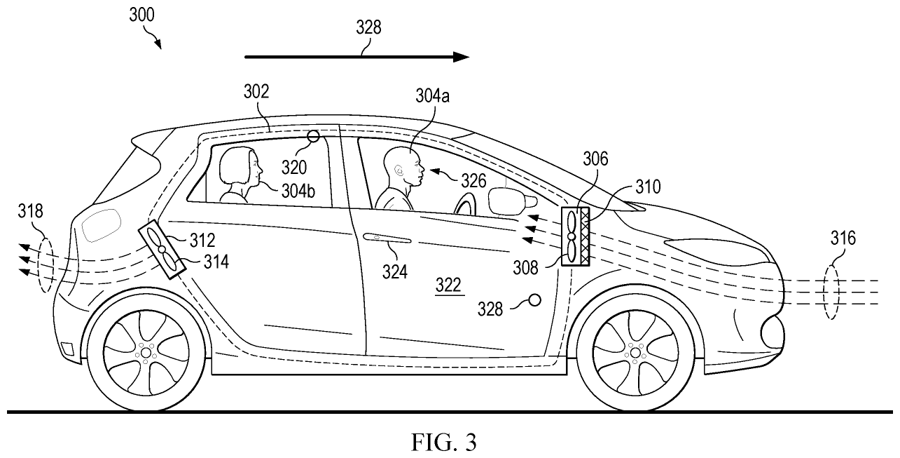

FIG. 3 shows a vehicle 300 with a vehicle cabin pressure system. The vehicle 300 includes a cabin 302 configured to seat a plurality of passengers 304 (e.g., 1-15 passengers). In an embodiment, vehicle 300 is the same as, or similar to, AV 100. The cabin 302 is the interior space within the vehicle 300 where the plurality of passengers 304 sit.

The vehicle 300 includes at least one door 322 with a locking mechanism 324. In an embodiment, the vehicle 300 includes at least one window 326 movably connected to the vehicle 300 and configured to move between an open state and a closed state. In an embodiment, each passenger of the vehicle 300 has a separate door 324 with a separate locking mechanism 324 and a separate window 326.

In general, the cabin 302 is at least partially sealed from the environment outside the vehicle 300. In some examples, door seals of the doors of the vehicle 300 at least partially seal the cabin 302. In some examples, air within the cabin 302 flows through door jambs, creases around windows, holes in the firewall, etc. In some examples, the air within the cabin 302 escapes into the environment due to a pressure gradient between the higher pressure of the cabin 302 and the lower pressure of the environment. For example, in some cases, the elastomeric door seals elastically deform to allow air within the cabin 302 to escape.

In an embodiment, the cabin 302 is hermetically sealed. In examples where the cabin 302 is sealed, air particles from outside the cabin 302 do not enter the cabin 302 and vice versa.

The vehicle 300 includes at least one inlet 306. In an embodiment, the inlet 306 is a vent or duct in a dashboard of the vehicle 300 and is configured to allow air to flow into and out of the cabin 302. The inlet 306 includes at least one inlet fan 308. In an embodiment, the inlet fan 308 is a blade-less blower. The inlet fan 308 causes air (generally shown by the air stream 316) that is (or has been) filtered to flow into the cabin 302 through the inlet 306.

The inlet fan 308 is controlled by at least one processor of the vehicle 300. For example, the processor controls the inlet fan 308 to turn on, turn off, speed up (e.g. 0 RPM to 5,000 RPM), or speed down (e.g., 5,000 RPM to 0 RPM), based on one or more conditions within the cabin 302 of the vehicle 300. In some examples, the speed of the fan 308 is greater than 5,000 RPM (e.g., 10,000 RPM). In some examples, the speed of the fan 308 is less than 5,000 RPM (e.g., 4,000 RPM).

In an embodiment, the vehicle 300 is moving, for example in a forward direction (i.e., along the direction 328) when the inlet fan 308 is controlled. In an embodiment, the vehicle 300 is stationary when the inlet fan 308 is controlled.

In an embodiment, the vehicle 300 includes at least one filter 310. In an embodiment, the filter 300 is placed adjacent to the inlet 306 such that the filter 300 filters, strains, purifies, cleanses, decontaminates, refines, or treats the air entering into cabin 302 by reducing or altering the quantity of particulates in the air entering cabin 302 (e.g., via the inlet fan 308). In an embodiment, the filter 300 is integrated into the inlet 306. In this way, air 316 flowing into the cabin 302 via the inlet 306 is filtered by passing the air through the filter 310.

In an embodiment, particulates include, but are not limited to, large dust particles (e.g., 100-1000 micrometers) to small virus particles (e.g. 0.001 to 0.5 micrometer). Particulates include ash, smoke, smog, soot, pollen, mold spores, allergens, and bacteria. For example, COVID-19 particles exist in a range of 0.06-0.14 micrometers. In some examples, the filter 310 is a 0.05 micrometer filter configured to filter out COVID-19 particles to limit or prevent the COVID-19 particles from entering the cabin 302. In an embodiment, the filter 310 is a high-efficiency particulate air (HEPA) filter.

In an embodiment, the vehicle 300 includes at least one outlet 312. In an embodiment, the outlet 312 includes at least one outlet fan 314. In an embodiment, the outlet fan 314 causes air (generally shown by the air stream 318) inside the cabin 302 to flow out of the cabin 302 through the outlet 312. In an embodiment, the outlet 312 is a vent or duct in a rear of the cabin 302. In an embodiment, the fan 314 is a blade-less blower.

The at least one outlet fan 314 is controlled by the processor of the vehicle 300. For example, in some examples, the processor controls the outlet fan 314 to turn on, turn off, speed up, or speed down, based on one or more conditions within the cabin 302 of the vehicle 300. In an embodiment, the inlet 306 is capable of being controlled so as to reverse the flow of air such that air flows out of cabin 302. In an embodiment, the outlet 312 is capable of being controlled so as to reverse the flow of air such that air flows out of cabin 302.

In an embodiment, the vehicle 300 includes at least one pressure sensor 320 configured to measure the pressure of the air within the cabin 302. The pressure sensor 302 is in communication with the processor of the vehicle 300 so that operations of the vehicle 300 are based on the received signal from the pressure sensor 320.

In some examples, the inlet fan 308 and the outlet fan 314 are controlled by the processor to cause a pressure of air within the cabin 302 to increase, decrease, or be maintained. In an embodiment, control of the inlet fan 308 and the outlet fan 314 are based on a received signal from the pressure sensor 320 indicating the pressure of air within the cabin 302.

In an embodiment, the pressure inside the cabin 302 is controlled by controlling the flow of air inside cabin 302. For example, the pressure inside cabin 302 can be increased above an ambient pressure of the environment of the vehicle 300 so that airborne particulates are forced out of the cabin 302 by a flow of air through the cabin 302 created by a pressure gradient between the air pressure inside the cabin 302 and the air pressure outside the cabin 302. In examples where the cabin 302 is sealed, the airborne particulates are removed from the cabin 302 using the outlet fan 314 and blowing air out of the cabin 302 (for example by moderating the speed of the outlet fan 314). In examples where the cabin 302 is not sealed, the airborne particles are forced out by the air escaping through the door jambs, creases around windows, holes in the firewall, etc.

The locking mechanism 324 is configured to lock and unlock the at least one door 322 in response to signals received from the processor of the vehicle 300. In some examples, the processor controls the locking mechanism 324 to lock the at least one door 322 when the ride of the vehicle 300 begins. In some examples, the processor controls the locking mechanism 324 to unlock the door 322 when the vehicle 300 halts or comes to a stop at the conclusion of a passenger ride (e.g., as determined from a ride completion signal).

In an embodiment, each passenger 304 has access to a separate door 322 for entry into the vehicle 300. In some examples, each separate door 322 is controlled by the processor based on one or more conditions of the passengers or anticipated passengers. In this context, an “anticipated passenger” is a passenger that is awaiting entry into the vehicle 300. In some examples, the door 322 closest to an anticipated passenger unlocks to allow entry of the anticipated passenger into the vehicle 300.

In an embodiment, the vehicle 300 includes a window sensor 328 configured to sense when the at least one window 326 is in a closed state. In some examples, each window of vehicle 300 has a window sensor 328 such that each window 326 is individually controllable by the processor of the vehicle 300.

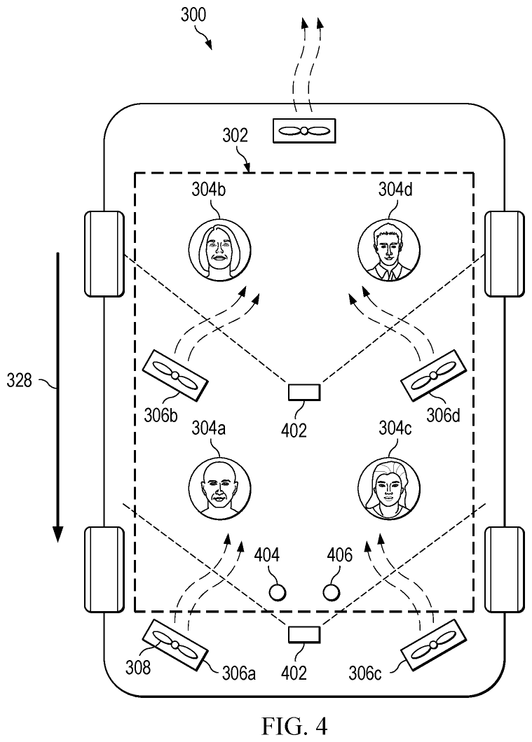

FIG. 4 shows a top schematic view of the vehicle 300 showing four passengers 304a-d, four inlets 306a-d, and a fan 308. The inlets 306a-d are arranged such that each inlet (e.g., each inlet of 306a, 306b, 306c, and 306d) is proximate to a passenger and each inlet is individually controllable by the processor of the vehicle 300 so that the airflow around each passenger is individually controllable by the processor of the vehicle 300.

In an embodiment, the processor of the vehicle 300 controls each inlet individually based on passenger seating arrangements (e.g., as detected using weight sensors throughout the cabin 302), passenger health (e.g., as detected using particulate sensors throughout the cabin 302), and/or passenger comfort settings (e.g., as defined from a user inquiry).

In an embodiment, each inlet is rotatable along its latitudinal axis in accordance with signals received from the processor of the vehicle 300 so the airflow can be directed through the cabin 302. In an embodiment, each inlet 306 is rotatable about an axis perpendicular to the ground of the vehicle 300.

In an embodiment, the vehicle 300 includes at least one occupancy sensor 402 configured to detect the number of passengers in the cabin 302. In some examples, the occupancy sensor 402 includes a system with two cameras arranged to detect the passengers in the front seats of the vehicle 300 and the passengers in the rear seats of the vehicle 300. In some examples, the camera images are transmitted to the processor of the vehicle 300 for facial recognition. In some examples, the signals from the occupancy sensor 402 are processed by the processor to determine the number of passengers within the vehicle 300.

In some examples, the at least one occupancy sensor 402 is a weight sensor or a pressure sensor built-into the seats of the vehicle 300. For example, when the occupancy sensor is a set of weight sensors configured to measure a weight of each passenger within the cabin 302 of the vehicle 300, the occupancy sensor sends a signal representing the weight and/or the number of passengers detected by the weight sensors to the processor of the vehicle 300.

In some examples, the at least one occupancy sensor 402 is an infrared (IR) camera configured to detect or measure the heat signature of the passengers. In some examples, the at least one occupancy sensor 402 is a LiDAR system configured to resolve the 3D position, velocity, and acceleration of the passengers.

In an embodiment, the vehicle 300 includes at least one particulate sensor 404 configured to measure a particulate level associated with the air inside the cabin 302. Examples of particulate sensors 404 include Honeywell HPM series particulate matter sensors and Sensirion SPS30 series particulate matter sensors. In some examples, the particulate sensor 404 provides information on the particle concentration or level for a given particle concentration range (e.g., a user defined or predetermined particle concentration range). In some cases, the information is transmitted to the processor via an air quality signal.

In some examples, the particulate level of the air inside the cabin 302 represents an amount of airborne bacteria within the cabin 302. In some examples, the particulate sensor 404 detects and counts particles in a concentration range of 0 μg/m3 to 1,000 μg/m3 within the cabin 302 of the vehicle 300. In some examples, the particulate level associated with the air inside the cabin 302 represents an amount of particles with a diameter of less than 0.05 micrometers included in the air inside the cabin 302.

In an embodiment, the vehicle 300 includes at least one thermal imaging sensor 406 configured to measure a body temperature of at least passenger 304 of the vehicle 300. In an embodiment, the thermal imaging sensor 406 is mounted on the dashboard of the vehicle 300. In an embodiment, the thermal imaging sensor 406 measures the body temperature of each of the passengers within the cabin 302.

In some examples, information from the thermal imaging sensor 406 is used by the processor to infer a passenger's (or anticipated passenger's) health. In some examples, if a passenger has a body temperature in the range of 100-102° F., the processor determines the passenger is unhealthy. In some examples, if a passenger has a body temperature in the range of 98-99° F., the processor determines the passenger is healthy. In some examples, a body temperature above 100° F. is used to infer that the passenger is unhealthy. In some examples, the thermal imaging sensor 406 images the passenger more than once (e.g., twice or three times). In some of these cases, the processor averages the measured body temperature. In some of these cases, the processor discards the highest measured body temperature to reduce false positives (i.e., measured temperature is higher than actual).

In an embodiment, the inlet 306 associated with the passenger who received an unhealthy assessment is controlled by the processor in response to receiving the unhealthy assessment. In some examples, the fan speed and the angle of airflow of the inlet 306 of the passenger who received the unhealthy assessment is controlled in response to receiving the unhealthy assessment.

In some examples, the processor controls the inlet 306 associated with the passenger who received the unhealthy assessment to run continuously (e.g., for up to one minute, for up to one hour, or for the entire direction of the vehicle trip). In these cases, continuously running the inlet 306 reduces the risk of spreading any airborne bacteria or illness from the passenger who received the unhealthy assessment to the other (presumably healthy) passengers.

In an embodiment, the thermal imaging sensor 406 is exterior facing so that an anticipated passenger outside the vehicle 300 is able to be imaged via the thermal imaging sensor 406 to determine the body temperature of the anticipated passenger before the passenger enters the vehicle 300. In an embodiment, the thermal imaging sensor 406 is mounted on the exterior of the vehicle 300. In some examples, the body temperature of the anticipated passenger is part of a health assessment to determine if the passenger should be granted entry into the vehicle 300.

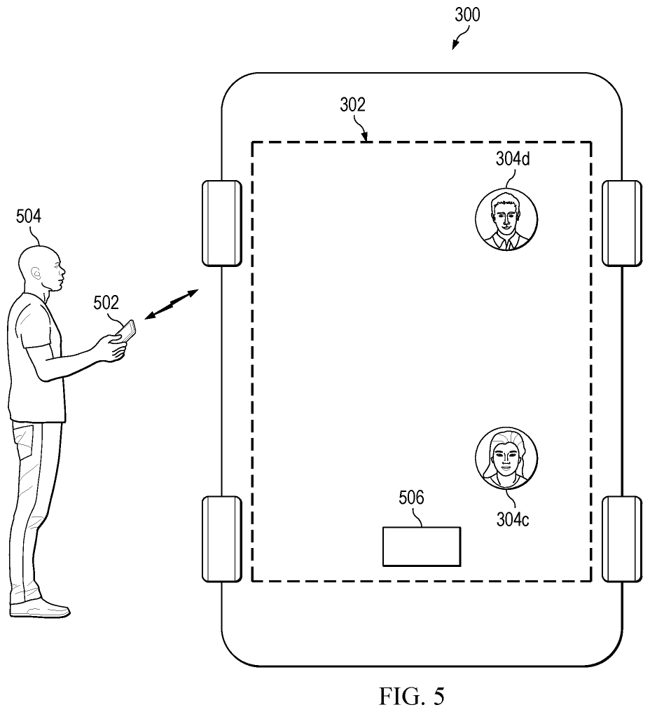

FIG. 5 shows the vehicle 300 in network communication (e.g., via cellular, 4G, 5G, Bluetooth, etc.) with a mobile device 502 (e.g., smartphone, tablet, smartwatch, etc.) of an anticipated passenger 504 of the vehicle 300. In some examples, the mobile device 502 is associated with one of the passengers 304c-d already within the vehicle 300.

In some examples, the mobile device 502 is configured to provide a health assessment inquiry via a touchscreen display of the mobile device 502 to the at least one passenger 504 before entering the vehicle 300. In some examples, the mobile device 502 transmits a response to the vehicle 300. In these examples, the health assessment presents a series of health questions and collects respective responses to determine whether the anticipated passenger 504 should be granted entry into the vehicle 300.

In some examples, the vehicle 300 denies entry (e.g., by not unlocking the vehicle doors) when the anticipated passenger indicates they have a fever or have travelled outside the country recently (e.g., in the past two weeks). In some examples, the health assessment includes one or more queries related to symptoms exhibited by the anticipated passenger 504, medical conditions, travel conditions, and/or known allergies that could be either aggravated by the air within the cabin 302 of the vehicle 300. In some examples, the heath assessment includes using the thermal camera 406 directed to the passenger's forehead to determine if the anticipated passenger 504 has a fever.

In some examples, the processor receives a passenger health signal representing whether the passenger has passed the health assessment. In this case, the processor controls the locking mechanism of a door of the vehicle 300 to unlock, thereby allowing the anticipated passenger entry into the vehicle 300. In some examples, the door closest to the anticipated passenger 504 is unlocked.

In an embodiment, a contact tracing database is queried to see if any passenger has been exposed to COVID-19 or other virus. If the passenger is found in the query results, the passenger is barred from entry into the vehicle 300 by, for example, locking the doors of vehicle 300. In an embodiment, vehicle 300 determines from a map when vehicle 300 is entering an area that has a high percentage of COVID-19 cases, and then automatically seals the vehicle 300 by rolling up the windows (e.g., after providing an audible warning to the passengers) and closing vents (e.g., the inlet 306 and the outlet 312) so that the air circulates within the vehicle 300. In some examples, when the vehicle 300 detects that it is operating in a rural or other area of low-density population, the vehicle 300 operates in an unsealed state (i.e., windows are open and outside vents (e.g., the inlet 306 and the outlet 312) are open). If, however, vehicle 300 detects that it is operating in a dense urban environment or approaching a traffic jam or other similar congested environment, the vehicle 300 automatically configures itself into a sealed state, where the windows are shut and the air is recirculated.

In some examples, the vehicle 300 detects whether it is operating in a rural or other area of low-density population vs. a dense urban environment or approaching a traffic jam or other similar congested environment based on receives signal from a perception system of the vehicle 300. For example, in some cases, the vehicle 300 acquires information of the environment around the vehicle 300 using LiDAR and camera sensors on board the vehicle 300 and determines a population or congestion metric based on the LiDAR and camera information. In some cases, the vehicle 300 receives the population or congestion metric from a perception module directly. In some examples, the population or congestion metric is downloaded from a map of the environment.

In some examples, the population or congestion metric is indicative of a number of people in the environment around the vehicle 300. For example, when no people are observed within a radius around the vehicle 300 (e.g., a 10, 20, 50, or 100 foot radius), the congestion metric is low (e.g., zero). In other examples, when more than ten people are observed within the same radius around the vehicle 300, the congestion metric is high (e.g., one).

In some examples, the population or congestion metric is indicative of a number of vehicles or traffic signals in the environment around the vehicle 300. For example, when no vehicles or traffic signals are observed within a radius around the vehicle 300 (e.g., a 10, 20, 50, or 100 foot radius), the congestion metric is low (e.g., zero). In other examples, when more than ten vehicles or traffic signals are observed within the same radius around the vehicle 300, the congestion metric is high (e.g., one).

In an embodiment, the vehicle 300 includes at least one display 506 (e.g., a touchscreen display) configured to provide a series of notifications to at least one of the passengers 304 within the vehicle 300. In an embodiment, the display 506 is mounted on the interior of the vehicle 300 so that is viewable by at least one passenger within the vehicle 300. In an embodiment, the display 506 is mounted on the exterior of the vehicle 300 so that is viewable by an anticipated passenger 504 before entering the vehicle 300. In some examples, the exterior display is configured to provide the health assessment inquiry to the anticipated passenger 504 before entering the vehicle 300.

In an embodiment, a passenger indicates a user comfort preference (via the at least one display 506 or via the mobile device 502) that is used by the vehicle 300 to determine what pressure level the passenger should be exposed to. For example, if a passenger indicates a sensitivity to pressures, the vehicle 300 uses this information to determine not to pressurize the cabin 302 when the passenger is within the vehicle 300. Likewise, if a passenger specifies that they are not affected by pressures, or does not know, the vehicle 300 uses this information to determine what pressure level to pressurize the cabin 302 to. If the passenger specifies an actual pressure limit (e.g., via a sliding scale), the vehicle 300 uses this information to limit the pressure in the cabin 302. Similarly, in some examples, if the passenger specifies a sensitivity to pressure and is experiencing an illness, the passenger is denied entry into the vehicle 300.

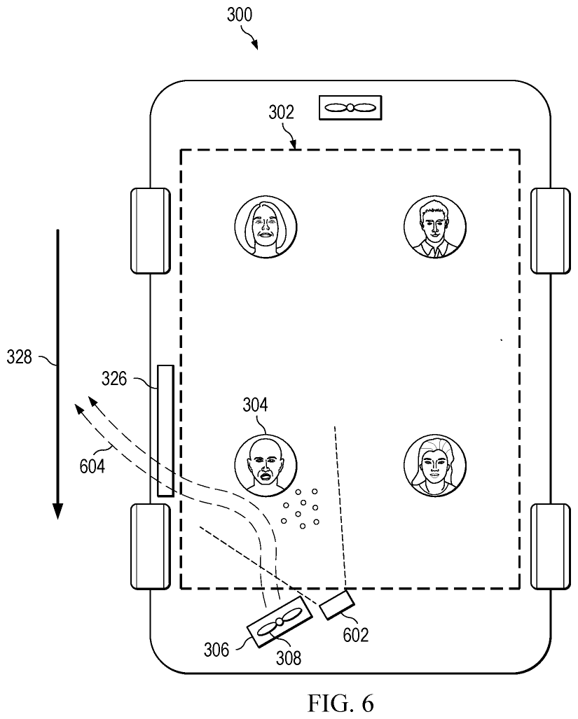

FIG. 6 shows the vehicle 300 with at least one audible sensor 602. The audible sensor 602 is configured to sense when at least one of the passengers 304 coughs or sneezes. In this way, the audible sensor 602 detects when particulates become airborne within the cabin 302. In an embodiment, audible sensor 602 continuously senses the sounds within the cabin 302 and transmits a signal representing this sound to the processor of the vehicle 300. In embodiment, the audible sensor 602 is at least two audible sensors 602 capable of sensing a source direction of sound.

The processor compares the received sounds from the audible sensor 602 with at least one database of sounds of people coughing and/or sneezing to determine a likelihood that the sound is a passenger coughing or sneezing. Once the likelihood reaches a threshold, the processor provides an indication (e.g., via the display 506 or via the display of the mobile device 506) that bacteria has become airborne within the cabin 302. In an embodiment, a neural network or other machine learning model is used to predict whether a particular passenger sound is a cough or sneeze.

In an embodiment, the inlet 306 is controlled in response to determining that the received sound is indicative of a passenger coughing or sneezing. In some examples, the fan 308 is turned on, turned off, sped up, or slowed down in response to determining that the received sound is indicative of a passenger coughing or sneezing. In some examples, an angle of airflow of the inlet 306 is rotated about a latitudinal axis using an electric motor in response to determining that the received sound is indicative of a passenger coughing or sneezing. In some examples, the processor controls the rotation of the inlet 306 to enable the inlet 306 to be directed at the passenger 306 so that airflow is directed to the passenger 306. In some examples, the processor controls the rotation of the inlet 306 to enable the inlet 306 to be directed toward the source of the sound emitted when the passenger coughed or sneezed.

In some examples, the processor controls the rotation of the inlet 306 to enable the inlet 306 to be directed toward the window 326 closest to the passenger 304 who coughed or sneezed and the processor controls the window 326 to open. In this scenario, the processor controls the fan 308 to turn on such that air is directed toward the window 326 to blow contaminated air (e.g., the air that contains the coughing or sneezing particulates) out of the cabin 302 (generally represented by the airstream 604).

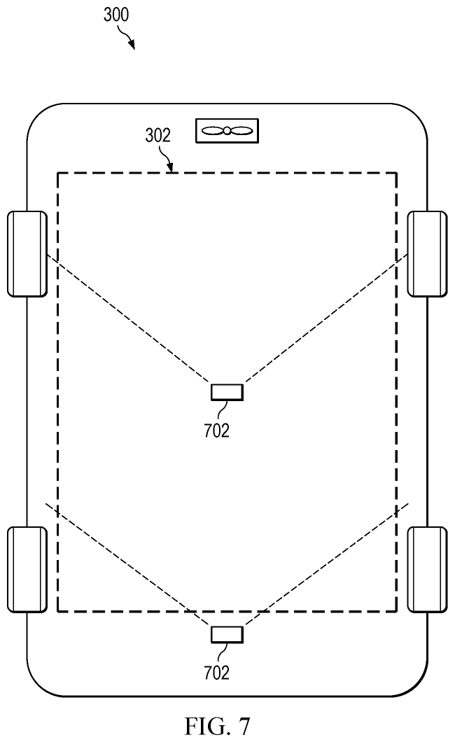

FIG. 7 shows the vehicle 300 with at least one ultraviolet light source 702. The ultraviolet light source 702 is configured to irradiate the air within the cabin 302 with ultraviolet light to reduce a bacteria level of the air within the cabin 302. In some examples, the ultraviolet light source 702 is controlled by the processor of the vehicle 300. In some examples, the processor turns on the ultraviolet light source 702 when zero passengers are detected within the cabin 302 (e.g., using the occupancy sensor 402 shown in FIG. 4). In some examples, the processor turns on the ultraviolet light source 702 when zero passengers are detected within the cabin 302 and when the air within the cabin 302 reaches a predetermined pressure threshold (e.g., 0.05 inches H₂O above ambient pressure) as measured or detected using the pressure sensor 320 (shown in FIG. 3). In some examples, the predetermined pressure threshold is greater than 0.05 inches H₂O above ambient pressure (e.g., 0.10 inches H₂O).

In an embodiment, the ultraviolet light source 702 irradiates the air within the cabin 302 with far-UVC light with a wavelength between 220 nm and 224 nm. In some examples, far-UVC light of 222 nm wavelength (i.e., between 220 nm and 224 nm) is used to reduce the bacteria level of the air within the cabin 302. Far-UVC light is safe for exposure to passengers within the vehicle 300. In some examples, the processor controls the ultraviolet light source 702 to irradiate the cabin 302 with far-UVC light with a wavelength between 220 nm and 224 nm when at least one passenger is present in the vehicle 300.

Referring back to FIG. 5, in some examples, a notification is pushed to the mobile device 502 of the anticipated passenger 504 of the vehicle 300. For example, when the anticipated passenger 504 requests a ride in the vehicle 300 using their mobile device 502, the mobile device 502 presents a notification that the vehicle 300 is either safe for entry or not safe for entry.

In some examples, the display 506 is configured to provide a first notification and a second notification representing safe and unsafe particulate levels within the vehicle 300, respectively. In some examples, the display 506 presents a first notification that the air within the cabin 302 is clean and void of dangerous bacteria or viruses and is safe for entry. In some examples, the display 506 presents a second notification that the air within the cabin 302 is not clean. In some examples, the display 506 presents a third notification that the air within the cabin 302 is currently being sanitized.

In an embodiment, route information of the vehicle 300 is used to determine a cleaning frequency (e.g., how often the cabin 302 is sanitized by the ultraviolet light source 702). In some examples, the ultraviolet light source 702 is activated every 5 minutes. In some examples, for trips of longer duration (e.g., 30 minutes-1 hour), the cabin 302 is sanitized every 20 minutes. In some embodiments, the sanitization frequency is based on the geographic location of the vehicle 300. For example, geographic locations associated with populous communities require increased sanitization frequency to reduce the spread of bacteria.

In an embodiment, the sanitization frequency is based on travel dates of the vehicle 300. In some embodiments, the sanitization frequency is based on weather conditions outside the vehicle 300. For example, the vehicle 300 will not open the window during cold temperatures or cold months of the year.

In an embodiment, the sanitization frequency and/or control parameters are based on the vehicle speed of the vehicle 300. In some examples, at least one window is opened during high-way speeds (e.g., above 40 MPH), to create a negative pressure in the cabin 302 to siphon airborne particulates within the cabin 302 out of the cabin 302. In another example, when the vehicle 300 is moving at a high rate of speed (e.g., above 40 MPH), the processor determines to allow the air that would normally be displaced by the moving vehicle 300, to be filtered, and enter the cabin 302.

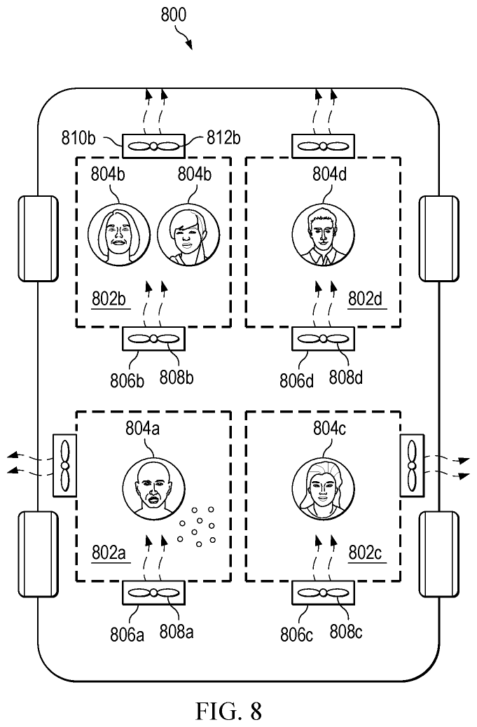

FIG. 8 shows a vehicle 800 with a cabin pressure system. The vehicle 800 includes a cabin with a plurality of compartments 802a-d. In an embodiment, vehicle 800 is similar to vehicle 300 or AV 100. In an embodiment, the cabin of the vehicle 800 includes at least two compartments 802. Each compartment 802a-d is configured to seat at least one passenger 804a-d plurality of passengers 304. In some examples, two passengers share a compartment (e.g., compartment 802b is shared by two passengers 804b). In an embodiment, the cabin includes separate compartments 802 for each passenger 804.

The cabin is compartmentalized (e.g., using plexiglass shielding) so that each passenger 804 of the vehicle 800 sits in a compartment 802 that is at least partially sealed from the other passengers 804. In some examples, each compartment 802 is sealed from the other compartments 802 and therefore passengers 804 within one compartment do not share air with passengers in another compartment. This is beneficial to isolate airborne particulates so the particulates are not shared amongst all passengers 804 of the vehicle 800.

In an embodiment, the cabin includes two compartments 802 (e.g., one for the front seat passengers 802a, 802c and one for the rear seat passengers 802b, 802d). In an embodiment, each compartment 802 includes the components previously described with reference to the single compartment cabin 302 of vehicle 300. For example, in an embodiment, each compartment 802 includes at least one inlet 806, at least one inlet fan 808, at least one outlet 810, and at least one outlet fan 812b. As a further example, in an embodiment, each compartment 802 includes any or all of the following components of vehicle 300: at least one pressure sensor (e.g., the same as, or similar to, similar to the pressure sensor 320), at least one occupancy sensor (e.g., the same as, or similar to, the occupancy sensor 402), at least one particulate sensor (e.g., the same as, or similar to, similar to particulate sensor 404), at least one thermal imaging sensor (e.g., the same as, or similar to, similar to the thermal imaging sensor 406), at least one display (e.g., the same as, or similar to, similar to the display 506, at least one audible sensor (e.g., the same as, or similar to, similar to the audible sensor 602), and at least one ultraviolet light source (e.g., the same as, or similar to, similar to the ultraviolet light source 702).

The processor of the vehicle 800 is connected to each inlet 806, each inlet fan 808, each outlet 810, each outlet fan 812b, each pressure sensor, each occupancy sensor, each particulate sensor, each thermal imaging sensor, each display, each audible sensor, and each ultraviolet light source.

In some examples, the processor of the vehicle 800 allocates a seat for the anticipated passenger if a sealed compartment with a single seat is available within the vehicle 800 when the anticipated passenger indicates that the anticipated passenger has symptoms of an illness (e.g., fever, cough, headache, runny nose, etc.) or has failed a health assessment.

In an embodiment, each seat in vehicle 800 includes a built-in powered air purifying respirator that can be connected to personal protective equipment (e.g., a ventilated Hazmat suit). This configuration of 800 could be used for workers traveling in hazardous areas.

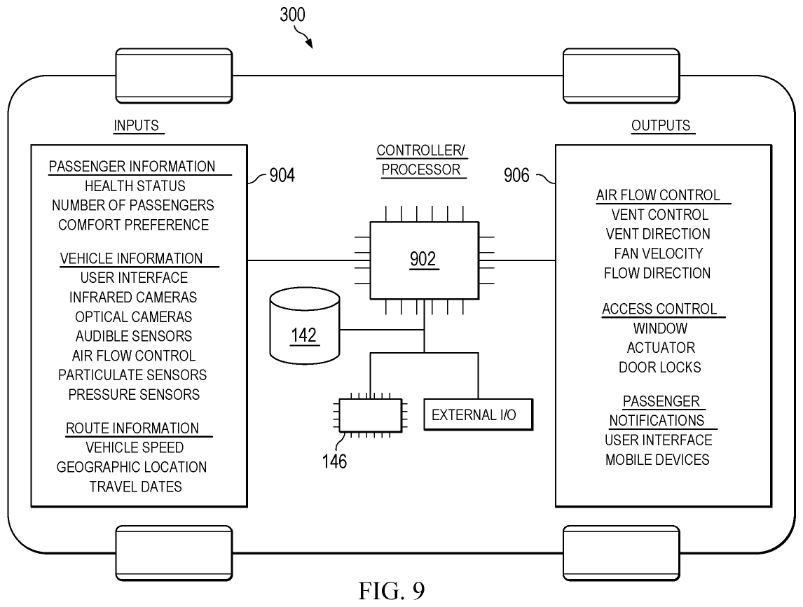

FIG. 9 shows a schematic of the components of a cabin pressure system of vehicle 300. The same or similar components are included in vehicle 800. The processor 902 receives inputs 904 from various sensors within the vehicle 300 (e.g., related to passenger information, vehicle information, and route information). The processor 902 determines control signals to control various aspects of the vehicle 300. In some examples, the processor 902 communicates with the other processors of the vehicle 300 (e.g., computer processors 146 described with reference to FIG. 1) and/or external devices (e.g., remote computer clusters, and/or mobile devices (e.g., mobile device 502.). In some examples, all of the control signals determined are determined off the vehicle 300 and transmitted back to the vehicle 300 for control. Once the control signals are determined, the processor 902 communicates these outputs 906 to control various aspects of the vehicle operation (e.g., air flow control, access control, passenger notification) over, for example a controller area network (CAN) bus, flexible data-rate CAN (FD-CAN) bus or Ethernet.

The vehicle 300 includes at least one non-transitory computer-readable media storing computer-executable instructions. The processor is communicatively coupled to the inlet fan, the occupancy sensor, and the computer-readable media. The processor is configured to execute the computer executable instructions such that operations are performed according to a first method 900 of the cabin pressure system.

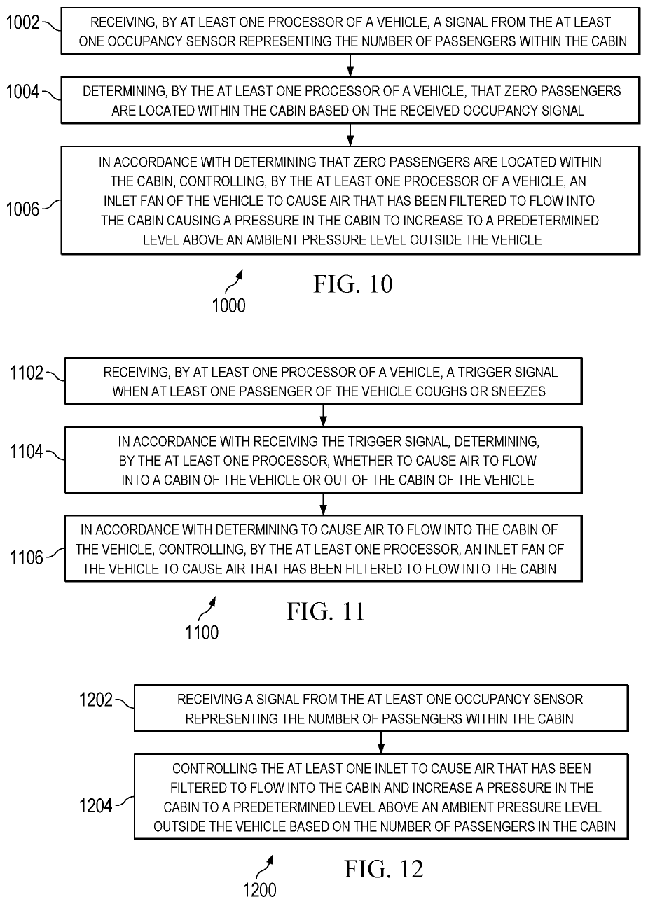

FIG. 10 shows a method 1000 of the cabin pressure system.

A signal is received 1002 by processor of a vehicle (e.g., the processor 902 of vehicle 300) from the occupancy sensor indicating the number of passengers within the cabin. In an embodiment, the processor determines 1004 that zero passengers are located within the cabin based on the received occupancy signal. In accordance with determining that zero passengers are located within the cabin, the processor controls 1006 an inlet fan of the vehicle to cause air that is (or has been) filtered to flow into the cabin causing a pressure in the cabin to increase to a predetermined level above an ambient pressure level outside the vehicle (e.g., where the predetermined level is 0.05 inches H₂O). In other words, when no passengers are inside the vehicle, the vehicle pressurizes the air within the cabin. For example, the controller turns the fans on or off depending on whether or not passengers are detected. In some examples, when no passengers are present, the vehicle performs a cleaning operation to cause pressure to rise in the cabin to a point where particulates within the cabin are forced out due to the high/low pressure gradient between the cabin air and the ambient air. In other embodiments, the pressurization process occurs regardless of whether passengers are within the vehicle.

In an embodiment, the processor receives a pressure signal representing a pressure within the cabin of the vehicle. In this case, the processor determines when the pressure represents the predetermined level using the received pressure signal representing a pressure within the cabin of the vehicle. In accordance with determining when the pressure represents the predetermined level, the processor controls the inlet fan of the vehicle to maintain the air flow such that the pressure in the cabin remains substantially constant (e.g., within a range of 0.01 inches H₂O).

In an embodiment, the processor receives an air quality signal representing a particulate level associated with the air inside the cabin of the vehicle. In this case, the processor determines that the particulate level is below a threshold (e.g., 50 μg/m³), based on the received air quality signal. In accordance with determining when the particulate level is below a threshold, the processor controls an outlet fan of the vehicle to cause air inside the cabin to flow out of the cabin. In some examples, the outlet fan of the vehicle is controlled to cause the pressure in the cabin to decrease to the ambient pressure level outside the vehicle. In some examples, the outlet fan of the vehicle is controlled to cause the pressure in the cabin to decrease below the ambient pressure level outside the vehicle. This occurs when the outlet fan is controlled to run to extract air from the cabin that is otherwise at ambient pressure levels. This creates a vacuum effect to siphon contaminated cabin air from cabin of the vehicle. In other words, once the particulate level reaches a safe level, the air pressure in the cabin is returned to ambient levels (e.g., in anticipation for passengers to enter the vehicle).

In an embodiment, in accordance with determining that the particulate level is below a threshold, the processor provides a first notification indicative of a safe particulate level of the vehicle and in accordance with determining that the particulate level is above a threshold, provides a second notification indicative of an unsafe particulate level of the vehicle. In some examples, provides the first notification is provided includes displaying a first indicator on a display of a mobile device. In some examples, providing the second notification includes displaying a second indicator on a display of a mobile device.

In an embodiment, the processor controls the inlet fan to maintain the air flow for at least one minute, thereby maintaining the pressure of the air inside the cabin of the vehicle at a pressure greater than the ambient pressure.

In an embodiment, the processor transmits safety data associated with an indication of whether the vehicle is safe for entry to a mobile device. In some examples, the safety data is configured to cause a display of the mobile device to provide a notification that the vehicle is safe for entry.

In an embodiment, the processor receives a ride completion signal representing whether a ride of the vehicle has completed. In other words, the ride completion signal indicates when the vehicle halts or comes to a stop at the conclusion of a passenger ride. In an embodiment, the processor controls the inlet fan of the vehicle based on the ride completion signal.

In an embodiment, the ultraviolet light source irradiates the air within the cabin with ultraviolet light to reduce a bacteria level of the air within the cabin. In an embodiment, the ultraviolet light source is controlled based on the received signal from the occupancy sensor.

In an embodiment, the processor of the vehicle receives a passenger health signal representing whether a passenger has passed a health assessment.

In an embodiment, the door of the vehicle nearest to the anticipated passenger is unlocked when the anticipated passenger has passed the health assessment (e.g., the vehicle lets the anticipated passenger into the vehicle only if the anticipated passenger passes the health assessment).

In an embodiment, in accordance with determining that zero passengers are in the vehicle, the processor controls a locking mechanism to lock each passenger door of the vehicle. In accordance with determining that the particulate level is below a threshold, the processor controls the locking mechanism to unlock each passenger door of the vehicle.

In an embodiment, a signal is received from the window sensor indicating that the window is in a closed state. In this case, controlling the inlet to cause air that is (or has been) filtered to flow into the cabin is based on the window being in the closed state. For example, if the window is closed, the processor determines that pressurizing the cabin is not possible and waits for the window to be closed before pressurizing the cabin.

In an embodiment, the inlet is controlled based on the body temperature of the at least one passenger. In an embodiment, the inlet is controlled based on the cough or sneeze of the at least one passenger.

In an embodiment, each passenger door of the vehicle is locked when the processor of the vehicle determines that zero passengers are in the vehicle (e.g., so that the cleaning operation can be performed). In an embodiment, each passenger door of the vehicle is unlocked when the processor of the vehicle determines that the particulate level is below a threshold. i.e., let them in when it is clean).

In an embodiment, the processor controls the inlet fan to maintain the air flow for at least one minute, thereby maintaining the pressure of the air inside the cabin of the vehicle at a pressure greater than the ambient pressure. In this case, once the air pressure within the cabin reaches the predetermined threshold, the inlet fan is slowed down. If the cabin pressure decreases, the fan speed increases. In this way, the air pressure within the cabin remains substantially constant (e.g., within a range of 0.01 inches H₂O) regardless of the leakage of pressure through the partial seal of the cabin.

In an embodiment, the processor of the vehicle transmits safety data associated with an indication of whether the vehicle is safe for entry to a mobile device when the particulate level is below a threshold. The safety data is configured to cause a display of the mobile device to provide a notification that the vehicle is safe for entry.

In an embodiment, the processor receives a ride completion signal representing whether a ride of the vehicle has completed; and controlling, by the processor, the inlet fan of the vehicle based on the ride completion signal (e.g., once the ride is over and passengers have exited the vehicle, the cabin is pressurized). In some examples, the vehicle is autonomous or non-autonomous vehicles).

FIG. 11 shows a second method 1100 of the cabin pressure system. In some examples, method 1100 is performed along with method 1000. In some examples, steps described with reference to method 1000 are used with method 1100.

The processor of a vehicle receives 1102 a trigger signal when at least one passenger of the vehicle coughs or sneezes. In accordance with receiving the trigger signal, the processor determines 1104 whether to cause air to flow into a cabin of the vehicle or out of the cabin of the vehicle. In accordance with determining to cause air to flow into the cabin of the vehicle, the processor controls 1106 the inlet fan of the vehicle to cause air that is (or has been) filtered to flow into the cabin.

In an embodiment, the processor controls the inlet fan of the vehicle to cause a pressure in the cabin to increase to a first predetermined level above an ambient pressure level outside the vehicle. In an embodiment, the processor controls the outlet fan of the vehicle to cause the pressure in the cabin to decrease to a second predetermined level below an ambient pressure level outside the vehicle.

In an embodiment, in accordance with determining to cause air to flow out of the cabin, the processor controls an outlet fan of the vehicle to cause cabin air to flow out of the cabin.

In an embodiment, the trigger signal is received from an audible sensor that detected the cough or sneeze of the passenger.

In an embodiment, the processor receives a window signal representing whether at least one window of the vehicle is open. In this case, the processor determines that the window is open based on the window signal. In accordance with determining that the window is open, the processor controls the inlet fan of the vehicle to cause air inside the cabin to exit the vehicle by displacing the air through the window.

In an embodiment, in accordance with not receiving the trigger signal, the processor controls the inlet fan of the vehicle to cause air that is (or has been) filtered to flow into the cabin.

In an embodiment, determining whether to cause air to flow into the cabin of the vehicle or out of the cabin of the vehicle is based on a ground speed of the vehicle.

In an embodiment, determining the predetermined pressure level of the cabin is based on a user comfort preference.

In an embodiment, the processor receives a bacteria signal representing a bacteria level within the cabin of the vehicle. In some examples, the processor determines that the bacteria level is below a threshold based on the received signal. In accordance with determining that the bacteria level is below the threshold, the processor provides a notification that the bacteria level of the vehicle is safe for the passenger.

FIG. 12 shows a third method 1200 of the cabin pressure system. In some examples, method 1200 is performed along with method 1000 and/or 1100. In some examples, steps described with reference to method 1000 and/or 1100 are used with method 1200.

A signal is received 1202 from at least one occupancy sensor of the vehicle representing the number of passengers within a cabin of the vehicle. The processor of the vehicle controls 1204 at least one inlet to cause air that is (or has been) filtered to flow into the cabin and increase a pressure in the cabin to a predetermined level above an ambient pressure level outside the vehicle based on a number of passengers in the cabin. For example, the cabin is configured to seat a plurality of passengers. The vehicle includes the inlet which includes at least one inlet fan. The inlet fan causes air that is (or has been) filtered to flow into the cabin through the at least one inlet.

In an embodiment, the processor controls at least one outlet fan to cause air inside the cabin to flow out of the cabin to cause a pressure in the cabin to decrease to the ambient pressure level outside the vehicle. For example, the outlet includes at least one outlet fan and the outlet fan causes air inside the cabin to flow out of the cabin through the outlet.

In an embodiment, the processor receives a signal from a particulate sensor representing the particulate level of the air inside the cabin. In some examples, controlling the inlet to cause air that is (or has been) filtered to flow into the cabin is based on the particulate level of the air inside the cabin. For example, the particulate sensor is configured to measure a particulate level associated with the air inside the cabin.

In an embodiment, the particulate level of the air inside the cabin represents an amount of airborne bacteria within the cabin. In an embodiment, the particulate level associated with the air inside the cabin represents an amount of particles with a diameter of less than 0.05 micrometers included in the air inside the cabin.

In an embodiment, the processor receives a signal from a window sensor indicating that at least one window is in a closed state. In some examples, controlling the inlet to cause air that is (or has been) filtered to flow into the cabin is based on the window being in the closed state. For example, the window of the vehicle is movably connected to the vehicle and configured to move between an open state and a closed state and a window sensor is configured to sense when the window is in the closed state.

In an embodiment, the processor controls the inlet based on a body temperature of the passenger. In some examples, the body temperature is measured by a thermal imaging sensor configured to measure a body temperature of at least one passenger of the passengers within the cabin of the vehicle.

In an embodiment, the processor controls the inlet based on a cough or sneeze of the passenger. For example, an audible sensor is configured to sense when at least one passenger of the passengers within the cabin of the vehicle coughs or sneezes.

In an embodiment, the processor controls an ultraviolet light source based on the received signal from the occupancy sensor. For example, the ultraviolet light source configured to irradiate the air inside the cabin with ultraviolet light to reduce a bacteria level of the air. In some examples, the ultraviolet light source is configured to irradiate the air inside the cabin with far-UVC light with a wavelength between 220 nm and 224 nm.

In an embodiment, the cabin includes at least two compartments and each of the at least two compartments is configured to seat at least one of the plurality of passengers.

In the foregoing description, embodiments of the invention have been described with reference to numerous specific details that may vary from implementation to implementation. The description and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction. Any definitions expressly set forth herein for terms contained in such claims shall govern the meaning of such terms as used in the claims. In addition, when we use the term “further comprising,” in the foregoing description or following claims, what follows this phrase can be an additional step or entity, or a sub-step/sub-entity of a previously-recited step or entity.

1. A vehicle comprising:
a cabin configured to seat a plurality of passengers;
at least one inlet comprising at least one inlet fan, wherein the at least one inlet fan causes air that has been filtered to flow into the cabin through the at least one inlet;
at least one occupancy sensor configured to detect a number of passengers in the cabin,
at least one computer-readable media storing computer-executable instructions; and
at least one processor communicatively coupled to the at least one inlet fan, the at least one occupancy sensor, and the computer-readable media, the at least one processor configured to execute the computer executable instructions, the execution carrying out operations including:
receiving a signal from the at least one occupancy sensor indicating the number of passengers within the cabin; and
controlling the at least one inlet to cause air that has been filtered to flow into the cabin and increase a pressure in the cabin to a predetermined level above an ambient pressure level outside the vehicle based on the number of passengers in the cabin.

2. The vehicle of claim 1, further comprising:
at least one outlet comprising at least one outlet fan, wherein the at least one outlet fan causes air inside the cabin to flow out of the cabin through the at least one outlet,
the operations further including:
controlling the at least one outlet fan to cause air inside the cabin to flow out of the cabin to cause a pressure in the cabin to decrease to the ambient pressure level outside the vehicle.

3. The vehicle of claim 1, further comprising:
a particulate sensor configured to measure a particulate level associated with the air inside the cabin,
the operations further including:
receiving a signal from the particulate sensor representing the particulate level of the air inside the cabin,
wherein controlling the at least one inlet to cause air that has been filtered to flow into the cabin comprises:
controlling the at least one inlet to cause air that has been filtered to flow into the cabin based on the particulate level of the air inside the cabin.

4. The vehicle claim 3, wherein the particulate level of the air inside the cabin represents an amount of airborne bacteria within the cabin.

5. The vehicle of claim 3, wherein the particulate level associated with the air inside the cabin represents an amount of particles with a diameter of less than 0.05 micrometers included in the air inside the cabin.

6. The vehicle of claim 1, further comprising:
at least one window movably connected to the vehicle and configured to move between an open state and a closed state; and
a window sensor configured to sense when the at least one window is in the closed state,
the operations further including:
receiving a signal from the window sensor indicating that the at least one window is in the closed state,
wherein controlling the at least one inlet to cause air that has been filtered to flow into the cabin comprises:
controlling the at least one inlet to cause air that has been filtered to flow into the cabin based on the at least one window being in the closed state.

7. The vehicle of claim 1, further comprising:
a thermal imaging sensor configured to measure a body temperature of at least one passenger of the passengers within the cabin of the vehicle; and
the operations further including:
controlling the at least one inlet based on the body temperature of the at least one passenger.

8. The vehicle of claim 1, further comprising:
an audible sensor configured to sense when at least one passenger of the passengers within the cabin of the vehicle coughs or sneezes; and
the operations further including:
controlling the at least one inlet based on the cough or sneeze of the at least one passenger.

9. The vehicle of claim 1, further comprising:
an ultraviolet light source configured to irradiate the air inside the cabin with ultraviolet light to reduce a bacteria level of the air; and
the operations further including:
controlling the ultraviolet light source based on the received signal from the at least one occupancy sensor.

10. The vehicle of claim 9, wherein the ultraviolet light source is configured to irradiate the air inside the cabin with far-UVC light with a wavelength between 220 nm and 224 nm.

11. The vehicle of claim 1, wherein the cabin includes at least two compartments, wherein each of the at least two compartments is configured to seat at least one of the plurality of passengers.

12. A method comprising:
receiving, by at least one processor of a vehicle, an occupancy signal indicating whether any passengers are located within a cabin of a vehicle;
determining, by the at least one processor, that zero passengers are located within the cabin based on the received occupancy signal; and
in accordance with determining that zero passengers are located within the cabin,
controlling, by the at least one processor, an inlet fan of the vehicle to cause air that has been filtered to flow into the cabin causing a pressure in the cabin to increase to a predetermined level above an ambient pressure level outside the vehicle.

13. The method of claim 12, further comprising:
receiving, by the at least one processor, a pressure signal representing the pressure within the cabin of the vehicle;
determining, by the at least one processor, that the pressure represents the predetermined level based on the received pressure signal; and
in accordance with determining that the pressure represents the predetermined level,
controlling, by the at least one processor, the inlet fan of the vehicle to maintain an air flow such that the pressure in the cabin remains within a range of 0.01 inches H₂O.

14. The method of claim 12, further comprising:
receiving, by the at least one processor, an air quality signal representing a particulate level associated with the air inside the cabin of the vehicle;
determining, by the at least one processor, that the particulate level is below a threshold based on the received air quality signal; and
in accordance with determining when the particulate level is below a threshold,
controlling, by the at least one processor, an outlet fan of the vehicle to cause air inside the cabin to flow out of the cabin.

15. (canceled)

16. The method of claim 14, wherein controlling the outlet fan of the vehicle to cause air inside the cabin to flow out the cabin causes the pressure in the cabin to decrease below the ambient pressure level outside the vehicle.

17. The method of claim 14, further comprising:
in accordance with determining that zero passengers are in the vehicle, locking each passenger door of the vehicle; and
in accordance with determining that the particulate level is below a threshold, unlocking each passenger door of the vehicle.

18. The method of claim 14, further comprising:
in accordance with determining that the particulate level is below the threshold, providing a first notification indicative of a safe particulate level of the vehicle; and
in accordance with determining that the particulate level is above the threshold, providing a second notification indicative of an unsafe particulate level of the vehicle,
wherein providing the first notification comprises displaying a first indicator on a display of a mobile device and providing the second notification comprises displaying a second indicator on the display of the mobile device.

19-20. (canceled)

21. The method of claim 17, further comprising:
receiving, by the at least one processor, a passenger health signal representing whether a passenger has passed a health assessment, wherein the health assessment includes at least one or more queries regarding whether the passenger has a fever or allergies; and
in accordance with determining when the particulate level is below the threshold and in accordance with receiving the passenger health signal representing when the passenger has passed the health assessment,
transmitting, by the at least one processor, safety data associated with an indication of whether the vehicle is safe for entry to a mobile device, wherein the safety data is configured to cause a display of the mobile device to provide a notification that the vehicle is safe for entry.

22. The method of claim 12, wherein the predetermined level is at least 0.05 inches H₂O above ambient pressure and controlling the inlet fan of the vehicle to cause air that has been filtered to flow into the cabin comprises:
controlling, by the at least one processor, the inlet fan to maintain an air flow for at least one minute, thereby maintaining the pressure of the air inside the cabin of the vehicle at the predetermined level of 0.05 inches H20 above ambient pressure for at least for at least one minute.

23-39. (canceled)

40. A non-transitory computer-readable storage medium comprising at least one program for execution by at least one processor of a first device, the at least one program including instructions which, when executed by the at least one processor, cause the first device to perform the method of:
receiving, by the at least one processor of a vehicle, an occupancy signal indicating whether any passengers are located within a cabin of a vehicle;
determining, by the at least one processor, that zero passengers are located within the cabin based on the received occupancy signal; and
in accordance with determining that zero passengers are located within the cabin,
controlling, by the at least one processor, an inlet fan of the vehicle to cause air that has been filtered to flow into the cabin causing a pressure in the cabin to increase to a predetermined level above an ambient pressure level outside the vehicle.
